(19) United States
(12) Patent Application Publication
(10) Pub. No.: US 2022/0125646 A1
(43) Pub. Date: Apr. 28, 2022

(54) COMPRESSION SOCK

(71) Applicant: CHUANG YUEH ENTERPRISE CO., LTD, Shetou Township (TW)

(72) Inventor: Chung-Hsing HSIAO, Shetou Township (TW)

(21) Appl. No.: 17/356,163

(22) Filed: Jun. 23, 2021

(30) Foreign Application Priority Data
Oct. 23, 2020 (TW) 109214009

(51) Int. Cl. A61F 13/08; A41B 11/00

(52) U.S. Cl. CPC A61F 13/08; D04B 1/265; A41B 11/005; A41B 11/003

(57) ABSTRACT

A compression sock includes a sock body made of a compression fabric which is formed by interweaving a plurality of first main and auxiliary yarns. Each first auxiliary yarn has a plurality of first yarn loops surrounding a corresponding first main yarn. The sock body includes a leg portion having a top end composed of a single layer elastic fabric which includes a plurality of interwoven second main and auxiliary yarns. Each second auxiliary yarn has a plurality of second yarn loops surrounding a corresponding second main yarn. A weaving density of the second yarn loops is smaller than that of the first yarn loops such that a binding pressure of the top end is smaller than that of the sock body.

### CROSS-REFERENCE TO RELATED APPLICATION

This application claims priority of Taiwanese Patent Application No. 109214009, filed on Oct. 23, 2020.

### FIELD

The disclosure relates to a hosiery, more particularly to a compression sock designed for the shape of left and right feet of a user.

### BACKGROUND

Compression socks are functional socks that can help to prevent or treat varicose veins in the legs of a user. The compression sock is usually worn from the sole to the lower leg of the user, the purpose of which is to apply appropriate pressure from the lower leg to urge the blood in the veins of the lower leg to flow to the thigh, reducing the problem of varicose veins. Thus, in the design of the compression sock, the pressure on the ankle is the greatest, and then the pressure decreases from the ankle to the lower leg, that is, the pressure reduces from bottom to top. On the other hand, due to high elasticity of the compression sock, a top end of the compression sock is pulled by a resilient force of the sock body during wear thereof, resulting in the top end of the compression sock being rolled and slid down from the top of the lower leg to the ankle. To prevent this phenomenon from happening, a top end of an existing compression sock has a double-layer fabric structure. However, because the top end of the existing compression sock has a double-layer fabric structure, the pressure at the top end thereof is greater than that at the lower leg, so that the pressure cannot be decreased from bottom to top during wear of the existing compression sock. Besides, in the existing compression socks, there is no distinction between left and right foot types. That is, the curve and curve length at the front ends of the existing compression socks corresponding to the toes of the left and right feet of the user are the same. When the existing compression socks are worn, the big toes of the feet of the user are compressed, resulting in discomfort during wear of the existing compression socks.

### SUMMARY

Therefore, an object of the present disclosure is to provide a compression sock that is capable of alleviating at least one of the drawbacks of the prior art.

Accordingly, a compression sock of this disclosure comprises a sock body made of a compression fabric. The compression fabric is formed by interweaving a plurality of first main yarns and a plurality of first auxiliary yarns. The first main yarns form weft threads of the compression fabric. The first auxiliary yarns form warp threads of the compression fabric. Each first auxiliary yarn has a plurality of first yarn loops surrounding a corresponding one of the first main yarns. The sock body includes a foot portion for covering a foot of the user, and a leg portion extending upwardly from the foot portion and configured to cover a lower leg of the user. The leg portion has a top end defining a foot opening for insertion of the foot of the user. The top end is composed of a single layer elastic fabric which includes a plurality of second main yarns and a plurality of second auxiliary yarns that are interwoven. The second main yarns form weft threads of the single layer elastic fabric. The second auxiliary yarns form warp threads of the single layer elastic fabric. Each second auxiliary yarn has a plurality of second yarn loops surrounding a corresponding one of the second main yarns.

The first and second main yarns are elastic covered yarns, and a weaving density of the second yarn loops is smaller than that of the first yarn loops such that a binding pressure of the top end of the leg portion is smaller than that of the sock body.

### BRIEF DESCRIPTION OF THE DRAWINGS

Other features and advantages of the disclosure will become apparent in the following detailed description of the embodiments with reference to the accompanying drawings, of which:

FIG. 1 is a right side view of a compression sock according to the first embodiment of the present disclosure;

FIG. 2 is a top view of the first embodiment;

FIG. 3 is an enlarged fragmentary diagrammatic view showing the relationship between a compression fabric and a single layer elastic fabric of the first embodiment;

FIG. 4 is a top view, illustrating another implementation aspect of the first embodiment; and

FIG. 5 is a right side view of a compression sock according to the second embodiment of the present disclosure.

### DETAILED DESCRIPTION

Before the present disclosure is described in greater detail with reference to the accompanying embodiments, it should be noted herein that like elements are denoted by the same reference numerals throughout the disclosure.

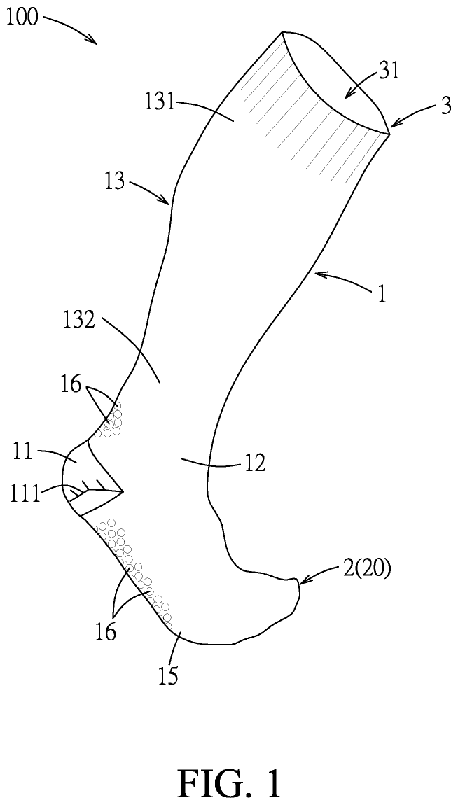

Referring to FIGS. 1 to 3, a compression sock 100 according to the first embodiment of the present disclosure comprises a sock body 1. The sock body 1 includes a foot portion 2, a leg portion 13, an ankle portion 12 between the foot portion 2 and the leg portion 13, and a plurality of three-dimensional woven structures 16.

The foot portion 2 includes a toe portion 20, a heel portion 11 opposite to the toe portion 20, and a sole portion 15 between the toe portion 20 and the heel portion 11. The leg portion 13 includes an upper part 131 having a top end 3, and a lower part 132 connected to the ankle portion 12.

When the compression sock 100 is worn on a foot of a user, the toe portion 20 corresponds to the toes of the user, the heel portion 11 corresponds to the heel of the user, the ankle portion 12 corresponds to the ankle of the user, the leg portion 13 corresponds to the lower leg of the user, and the sole portion 15 corresponds to the sole of the user.

The sock body 1 is made of a compression fabric 40 which can be any type of knitted fabric, mainly nylon and spandex fabrics. In this embodiment, the compression fabric 40 is formed by interweaving a plurality of first main yarns 401 and a plurality of first auxiliary yarns 402. The first main yarns 401 form the weft threads of the compression fabric 40, while the first auxiliary yarns 402 form the warp threads thereof. Each first auxiliary yarn 402 has a plurality of first yarn loops 403 surrounding a corresponding one of the first main yarns 401. Specifically, the sock body 1 is a woven structure that is formed by weaving the compression fabric 40 using a weaving machine and that conforms to the shape of the foot of the user. A weaving density of the first yarn loops 403 sequentially gradually decreases from the ankle portion 12 to the lower part 132 and the upper part 131 of the leg portion 13 so as to sequentially gradually reduce a binding pressure of the sock body 1 from the ankle portion 12 to the lower part 132 and the upper part 131 of the leg portion 13. In this embodiment, the binding pressure at the lower part 131 of the leg portion 13 is decreased by 70-80% relative to that at the ankle portion 12, while the binding pressure at the upper part 131 of the leg portion 13 is decreased by 50-60% relative to that at the ankle portion 12. Through this, blood can effectively flow upward when the compression sock 100 is worn.

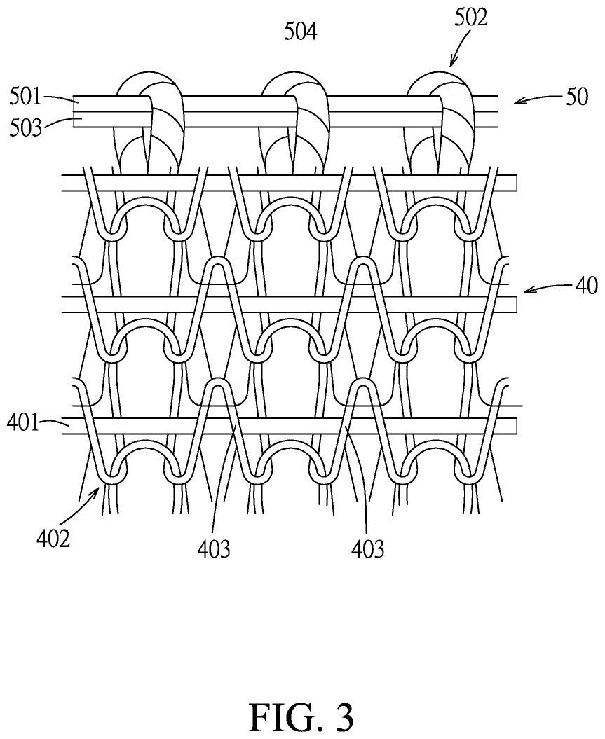

With reference to FIGS. 1 and 3, it should be noted herein that, in comparison with the existing compression sock which has only one short curved structure (not shown) at a heel portion thereof, the heel portion 11 of the compression sock 100 has a plurality of interconnected Y-shaped woven structures 111. In this embodiment, three Y-shaped woven structures 111 are exemplified in this embodiment, but is not limited thereto, and an extending length of the interconnected Y-shaped woven structures 111 is greater than the length of the short curved structure of the existing compression sock. Specifically, from the side view of FIG. 1, it can be seen that the heel portion 11 has a fan shape, so that the fabric required for the heel portion 11 that is adjacent to the sole of the foot is greater than the fabric required for the heel portion 11 that is adjacent to the back of the foot. Similarly, from the side view of FIG. 1, it can be seen that the Y-shaped woven structures 111 are arranged such that top ends of the Y-shaped woven structures 111 are facing the arc of the fan-shaped heel portion 11, so that the fabric required for the heel portion 11 that is adjacent to the sole of the foot is greater than the fabric required for the heel portion 11 that is adjacent to the back of the foot. Thus, the heel portion 11 can present a three-dimensional effect and can conform with the curvature of the heel of the user, thereby improving the comfort of wearing the compression sock 100. On the other hand, each Y-shaped woven structure 111 can be stretched in the direction of the ankle portion 12 and the leg portion 13 at the same time, so that, when the compression sock 100 is worn on the foot of the user, the heel portion 11 can be stretched corresponding to the shape of the heel of the user, increasing the comfort of wearing the compression sock 100.

Each of the three-dimensional (3D) woven structures 16 has four interwoven two-ply nylon yarns (not shown) forming the weft and warp threads thereof, so that each 3D woven structure 16 has a thickness greater than that of the compression fabric 40, and protrudes outward relative to the same. In this embodiment, the 3D woven structures 16 are configured as an array of bumps, and are provided on the ankle portion 12 and the sole portion 15 of the compression sock 100, as shown in FIG. 1. The array of bumps of the 3D woven structures 16 provided on the ankle portion 12 can thicken the fabric thereof to enhance the protective effect of Achilles tendon, and the array of bumps of the 3D woven structures 16 provided on the sole portion 15 can increase the friction of the sole portion 15 to improve the anti-slip function of the compression sock 100.

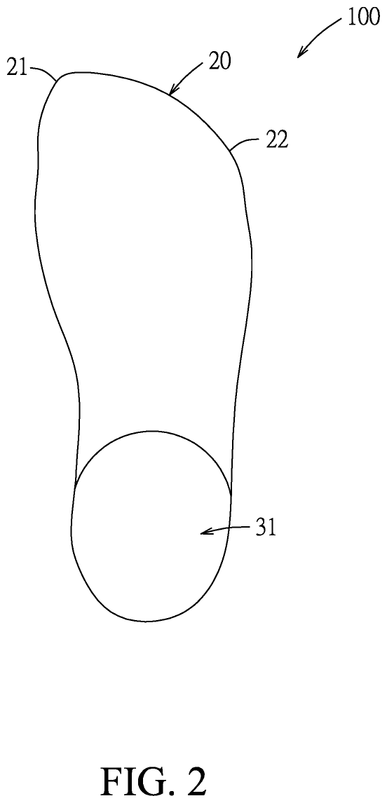

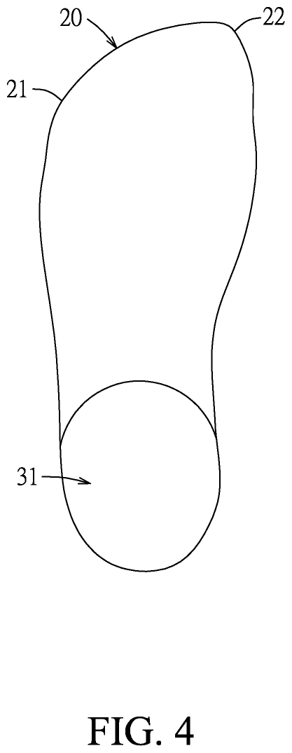

With reference to FIGS. 1, 2 and 4, the toe portion 20 has a left curved part 21 and a right curved part 22 that are asymmetric. In an implementation, as shown in FIG. 2, the left curved part 21 protrudes more forward than the right curved part 22, so that the left curved part 21 includes a big toe part, while the right curved part 22 includes a little toe part. Hence, the compression sock 100 shown in FIG. 2 is suitable to be worn by the right foot of the user. In another implementation, as shown in FIG. 4, the right curved part 22 protrudes more forward than the left curved part 21, so that the right curved part 22 includes the big toe part, while the left curved part 21 includes the little toe part. Hence, the compression sock 100 shown in FIG. 4 is suitable to be worn by the left foot of the user. Through appropriate configuration of the left and right curved parts 21, 22, the compression sock 100 can be worn in conformity with the foot shape, thereby increasing the comfort of wearing the compression sock 100.

With reference to FIGS. 1 and 3, the top end 3 of the upper part 131 of the leg portion 13 defines a foot opening 31 for insertion of the foot of the user. The top end 3 is composed of a single layer elastic fabric 50. The single layer elastic fabric 50 includes a plurality of second main yarns 501, a plurality of third main yarns 503 extending in a same direction as the second main yarns 501, and a plurality of second auxiliary yarns 502 interweaving with the second and third main yarns 501, 503. The second and third main yarns 501, 503 cooperatively form the weft threads of the single layer elastic fabric 50, while the second auxiliary yarns 502 form the warp threads thereof. Each second auxiliary yarn 502 has a plurality of second yarn loops 504 surrounding one of the second main yarns 501 and a corresponding one of the third main yarns 503. Specifically, the top end 3 is a single layer ribbed top woven from a single layer elastic fabric using a weaving machine.

It should be noted herein that the first and second main yarns 401, 501 are elastic covered yarns that can make the compression fabric 40 and the single layer elastic fabric 50 to have the advantages of easy stretching and good elastic recovery so as to achieve the purpose of wearing the compression sock 100 comfortably and preventing the compression sock 100 from easily sliding down. Simultaneously, the weaving density of the second yarn loops 504 is smaller than that of the first yarn loops 403, so that the binding pressure of the top end 3 is smaller than that of the sock body 1, thereby making the top end 3 more stretchable during wear of the compression sock 100. Further, in coordination with the advantages of easy stretching and good elastic recovery of the compression fabric 40 and the single layer elastic fabric 50, the compression sock 100 is suitable for users having different calf circumferences. For example, the calf circumference of an adult male is 38 cm. The conventional compression sock is suitable for leg with a calf circumference of 38 cm to 53 cm, whereas the compression sock 100 of this disclosure is suitable for calf circumference of 38 cm to 63 cm. Thus, the compression sock 100 is suitable for use in a wider range of calf circumference.

Moreover, in this embodiment, the weaving density of the second yarn loops 504 is, for example, 50% of the weaving density of the first yarn loops 403, the effect of the binding pressure of the top end 3 being less than that of the sock body 1 can be achieved. It is worth to mention herein that, in this embodiment, the third main yarns 503 may be one of cotton yarn, nylon yarn, wool yarn, and other functional yarns, so that the third main yarns 503 are softer than the second main yarns 501, thereby increasing the comfort at the top end 3 of the leg portion 13 during wear of the compression sock 100. In this embodiment, each of the first and second auxiliary yarns 402, 502 may be woven from one of single covered yarn and nylon yarn and one of cotton yarn, nylon yarn, wool yarn and other functional yarns.

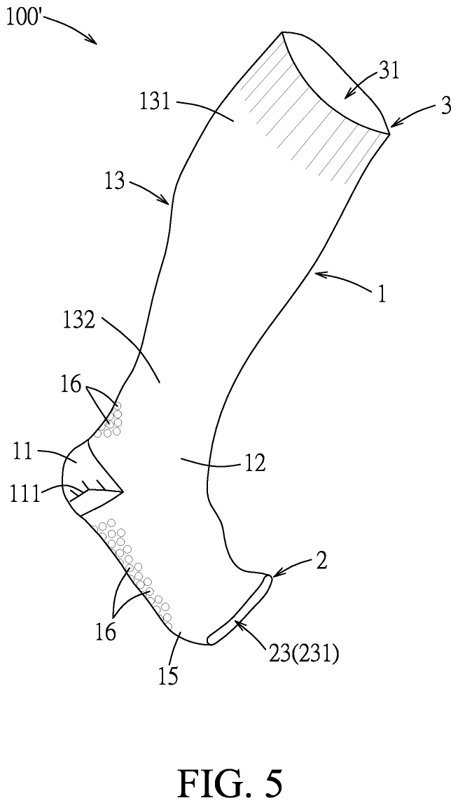

Referring to FIG. 5, a compression sock 100′ according to the second embodiment of the present disclosure is substantially similar to the first embodiment, and differs in that, in the second embodiment, the foot portion 2 has a cutaway portion 23 defining a toe opening 231 through which toes of the user can extend. That is, the toe portion 20 (see FIG. 1) of the foot portion 2 is dispensed herewith. The cutaway portion 23 is composed of a single layer elastic fabric for tightening the toes of the user, so that the cutaway portion 23 is bound to the front end of the foot of the user and does not easily retract into the sole portion 15. The compression sock 100′ of this embodiment can similarly conform to the shape of the foot of the user and can provide increased comfort during use thereof.

In summary, in the compression sock 100, 100′ of this disclosure, with the top end 3 of the upper part 131 of the leg portion 13 being made of the single layer elastic fabric 50, and with the first main yarns 401 of the single layer elastic fabric 50 being elastic covered yarns, the top end 3 of the leg portion 13 can be bound to the leg of the user through the resiliency of the single layer elastic fabric and does not need to be formed into a double layer fabric, so that the purpose of preventing the top end 3 of the leg portion 13 from easily sliding down along the leg of the user can be achieved. Further, with the weaving density of the second yarn loops 504 being smaller than that of the first yarn loops 403, and with the weaving density of the first yarn loops 403 sequentially gradually reducing from the ankle portion 12 to the lower part 132 and the upper part 131 of the leg portion 13, the binding pressure of the compression sock 100, 100′ can sequentially gradually decrease from the ankle portion 12 to the lower part 132, the upper part 131 and the top end 3 of the leg portion 13, thereby achieving the purpose of decreasing the binding pressure of the compression sock 100, 100′ from bottom to top. Moreover, with one of the left and right curved parts 21, 22 of the toe portion 20 including the big toe part, and with the other one of the left and right curved parts 21, 22 of the toe portion 20 including the little toe part, the compression sock 100 can be worn in conformity with the foot shape, thereby increasing the comfort during wear of the compression sock 100. It is worth to mention herein that, through the foot opening 231 in the cutaway portion 23 of the foot portion 2, the toes of the user can extend out of the foot opening 231 during wear of the compression sock 100′, so that the compression sock 100′ can similarly be worn in conformity with the foot shape. Therefore, the object of this disclosure can indeed be achieved.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A compression sock comprising:
a sock body made of a compression fabric, said compression fabric being formed by interweaving a plurality of first main yarns and a plurality of first auxiliary yarns, said first main yarns forming weft threads of said compression fabric, said first auxiliary yarns forming warp threads of said compression fabric, each of said first auxiliary yarns having a plurality of first yarn loops surrounding a corresponding one of said first main yarns, said sock body including
a foot portion for covering a foot of the user; and
a leg portion extending upwardly from said foot portion and configured to cover a lower leg of the user, said leg portion having a top end that defines a foot opening for insertion of the foot of the user, said top end being composed of a single layer elastic fabric, said single layer elastic fabric including a plurality of second main yarns and a plurality of second auxiliary yarns that are interwoven, said second main yarns forming weft threads of said single layer elastic fabric, said second auxiliary yarns forming warp threads of said single layer elastic fabric, each of said second auxiliary yarns having a plurality of second yarn loops surrounding a corresponding one of said second main yarns;
wherein, said first main yarns and said second main yarns are elastic covered yarns, and a weaving density of said second yarn loops is smaller than a weaving density of said first yarn loops such that a binding pressure of said top end of said leg portion is smaller than a binding pressure of said sock body.

2. The compression sock as claimed in claim 1, wherein said foot portion includes a toe portion, said toe portion having a left curved part and a right curved part that are asymmetric, one of said left curved part and said right curved part including a big toe part, the other one of said left curved part and said right curved part including a little toe part.

3. The compression sock as claimed in claim 1, wherein said foot portion has a cutaway portion defining a toe opening through which toes of the user can extend, said cutaway portion being composed of a single layer elastic fabric for tightening the toes of the user.

4. The compression sock as claimed in claim 1, wherein said single layer elastic fabric of said top end of said leg portion further includes a plurality of third main yarns extending in a same direction as said second main yarns, said third main yarns and said second main yarns cooperatively forming the weft threads of said single layer elastic fabric, said second yarn loops of each of said second main yarns surrounding one of said second main yarns and a corresponding one of said third main yarns, said third main yarns being softer than said second main yarns.

5. The compression sock as claimed in claim 1, wherein said foot portion includes a heel portion having a plurality of interconnected Y-shaped woven structures.

6. The compression sock as claimed in claim 5, wherein said sock body further includes an ankle portion between said foot portion and said leg portion, said leg portion including an upper part having said top end, and a lower part connected to said ankle portion, said weaving density of said first yarn loops sequentially gradually decreasing from said ankle portion to said lower part and said upper part of said leg portion so as to sequentially gradually reduce the binding pressure of said sock body from said ankle portion to said lower part and said upper part of said leg portion.

7. The compression sock as claimed in claim 6, wherein said foot portion further includes a sole portion connected to said heel portion, said sock body further including a plurality of three-dimensional woven structures provided on at least one of said ankle portion and said sole portion, each of said three-dimensional woven structures having four interwoven two-ply nylon yarns forming weft and warp threads thereof, each of said three-dimensional woven structures having a thickness greater than that of said compression fabric, and protruding outward relative to said compression fabric.

8. The compression sock as claimed in claim 7, wherein said three-dimensional woven structures are configured as an array of bumps.
